(19) United States
(12) Patent Application Publication
(10) Pub. No.: US 2021/0069071 A1
(43) Pub. Date: Mar. 11, 2021

(54) COSMETIC SOAP GEL STICKS WITH IMPROVED MANUFACTURABILITY

(71) Applicant: Henkel AG & Co. KGaA, Duesseldorf (DE)

(72) Inventors: INGA KERSTIN VOCKENROTH, Duesseldorf (DE); CLEMENS RIECKE, Remscheid (DE)

(73) Assignee: Henkel AG & Co. KGaA, Duesseldorf (DE)

(21) Appl. No.: 17/007,884

(22) Filed: Aug. 31, 2020

(30) Foreign Application Priority Data
Sep. 5, 2019 (DE) 10 2019 213 528.5

(51) Int. Cl. A61K 8/02; A61K 8/89; A61K 8/34; A61Q 19/10; A61Q 5/02; A61K 8/36; A61K 8/37

(52) U.S. Cl. CPC A61K 8/0229; A61K 8/89; A61K 8/345; A61K 8/375; A61Q 5/02; A61K 8/361; A61Q 19/10

(57) ABSTRACT

A cosmetic stick composition comprises
a) the sodium salt of at least one C₁₀-C₃₀ alkanecarboxylic acid, which may be substituted with at least one hydroxyl group, in a total amount of 5-7% by weight, based on the total composition,
b) at least one polyol liquid at 20° C., chosen from polyvalent C₂-C₉ alkanols with 2-6 hydroxyl groups and polyethylene glycol with 3-10 ethylene oxide units and mixtures thereof, in a total amount of 60 to 80% by weight, based on the total composition,
c) 10-35% by weight, preferably 15-30% by weight of water, based on the total composition, and
d) at least one linear polydimethylsiloxane which is liquid at 20° C. in a total quantity of 0.005-0.15% by weight, based on the total composition.

### CROSS-REFERENCE TO RELATED APPLICATION

This application claims priority to German Patent Application No. 10 2019 213 528.5, filed Sep. 5, 2019, which is incorporated herein by reference in its entirety.

### TECHNICAL FIELD

The present application concerns a shaped preparation for applying cosmetic and pharmaceutical active ingredients to the skin and/or hair, in particular in stick form, which is in the form of a gel based on a fatty acid salt, that is to say, a soap, has good dimensional stability and can be easily manufactured.

### BACKGROUND

Soap gels have long been known as carriers for topically applied cosmetic and pharmaceutical active ingredients. Usually sodium stearate gels are used as soap gels in mixtures of water with polyols, such as 1,2-propanediol or dipropylene glycol. Mixtures of water and monohydric C₁-C₄ alcohols, such as ethanol, are also suitable, but have disadvantages in terms of skin compatibility and storage stability and are rarely used nowadays. In addition, attempts have been made to incorporate skin-softening or refatting components into soap-gel pens. This leads to inhomogeneity and separation of the known water-insoluble oil components. For this reason, water-soluble or water-dispersible emollients have always been used, e.g. according to EP 450 597 A2 fatty alcohol-alkylene oxide adducts. However, these emollients do not show sufficient cosmetic and skin caring effects.

In addition, in the large-scale production of soap-gel bars the problem arises that their foaming properties can hinder and greatly delay product filling.

EP 823 252 A2 reveals anhydrous soap gel sticks containing from about 35-45% by weight of the volatile silicone oil cyclomethicone or decamethylpentasiloxane, from about 10-22% by weight of a polyol and a content of film-forming polymers suitable for application on the hair, especially as styling product. Soap-gel pens with such a high silicone oil content and because of the low concentration of hydrophilic polyol phase and the absence of water, the problem of foaming during filling does not arise. In addition, the use of cyclomethicone, such as decamethylpentasiloxane, is not appreciated by some consumer groups, as these are synthetic oils which are only slowly degraded in the environment.

### BRIEF SUMMARY

This disclosure provides a cosmetic stick composition in the form of a gel based on a fatty acid salt and comprising

the sodium salt of at least one C₁₀-C₃₀ alkanecarboxylic acid, which may be substituted with at least one hydroxyl group, in a total amount of from about 5-7% by weight, based on the total weight of the composition,

at least one polyol that is liquid at about 20° C., chosen from polyvalent C₂-C₉ alkanols with 2-6 hydroxyl groups and polyethylene glycol with 3-10 ethylene oxide units and mixtures thereof, in a total amount of from about 60 to about 80% by weight, based on the total weight of the composition,

from about 10-35% by weight of water, based on the total weight of the composition, and

at least one linear polydimethylsiloxane which is liquid at about 20° C. in a total quantity of from about 0.005-0.15% by weight based on the total weight of the composition.

In another embodiment, this disclosure provides a cosmetic stick composition in the form of a gel based on a fatty acid salt and comprising:

a sodium salt of a mixture of palmitic acid and stearic acid in a weight ratio of about 50:50 and present in a total amount of about 5-7% by weight, based on the total composition;

a mixture of 1,2-propanediol and dipropylene glycol comprising about 60-40% by weight of dipropylene glycol and present in a total amount of about 60 to 80% by weight based on the total weight of the composition;

15-30% by weight of water based on the total weight of the composition;

at least one linear polydimethylsiloxane which is liquid at 20° C. in a total quantity of about 0.04-0.07% by weight based on the total weight of the composition;

one or more nonionic oil-in-water emulsifiers chosen from glyceryl monoalkyl esters, glyceryl monoalkenyl esters, glyceryl monoalkyl ethers and glyceryl monoalkenyl ethers whose alkyl radical or alkenyl radical has at least 6 carbon atoms and that is present in a total amount of about 0.5-1% by weight based on the total weight of the composition;

at least one deodorant active substance and/or at least one fragrance substance chosen from 2-ethylhexyl glycerol ethers, hexachlorophene, triclosan (2,4,4′-trichloro-2′-hydroxydiphenyl ether), 3,4,4′-trichlorocarbanilide, chlorhexidine (1,1′-hexamethylene-bis-[5-(4-chlorophenyl)]-biguanide), chlorhexidine gluconate, benzalkonium halides, cetylpyridinium chloride, sodium bicarbonate, sodium phenolsulphonate, zinc phenolsulphonate, 1,2-alkane-(C₆-C₁₈)-diols, phenoxyethanol, phenoxyisopropanol (3-phenoxy-propan-2-ol), aniseed alcohol, 2-methyl-5-phenyl-pentan-1-ol, 1,1-dimethyl-3-phenyl-propan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2.2-dimethyl-3-phenylpropan-1-ol, 2.2-dimethyl-3-(3′-methylphenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-oll, 2-ethyl-3-(2′-methylphenyl)-propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3′,4′-dimethoxyphenyl)-2-ethylpropan-1-ol, 2-allyl-3-phenylpropan-1-ol and 2-n-pentyl-3-phenylpropan-1-ol, and mixtures of these active substances;

at least one polypropylene glycol with 26 propylene oxide units in a total amount of about 0.1-0.15% by weight based on the total weight of the composition;

at least one polyethylene glycol/polypropylene glycol copolymer having about 15-25 ethylene oxide units and about 40-80 propylene oxide units in the molecule in an amount of about 0.07-0.1% by weight based on the total weight of the composition; and

at least one trimethylsiloxysilicate/dimethicone crosspolymer in an amount of about 0.005-0.01% by weight based on the total weight of the composition.

The purpose of the present disclosure was to provide a stable pencil composition in the form of a gel based on a fatty acid salt, i.e. soap, with improved industrial-scale manufacturability. The use of cyclic silicones should be avoided wherever possible. Another task was to provide effective deodorant compositions in the form of stable soap-gel sticks. As many deodorant active ingredients are hydrophilic and their effectiveness could be impaired by the addition of hydrophobic ingredients, soap gel bars should contain as few hydrophobic ingredients as possible, especially oils.

Surprisingly, it was found that the task set can be solved by adding an exceedingly small amount of a linear polymethylsiloxane liquid at about 20° C. to a soap gel composition containing higher amounts of a polyol/water mixture. It is particularly surprising that even a total amount of from about 0.005 to a maximum of about 0.15% by weight of linear polymethylsiloxane liquid at about 20° C., based on the entire composition, has a strong antifoaming effect.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The subject of the present application is therefore a cosmetic stick composition in the form of a gel based on a fatty acid salt, that is to say, a soap, and

a) the sodium salt of at least one C₁₀-C₃₀ alkanecarboxylic acid, which may be substituted with at least one hydroxyl group, in a total amount of from about 5-7% by weight, based on the total composition, further
b) at least one polyol liquid at about 20° C., chosen from polyvalent C₂-C₉ alkanols with 2-6 hydroxyl groups and polyethylene glycol with 3-10 ethylene oxide units and mixtures thereof, in a total amount of from about 60 to about 80% by weight, based on the total composition, furthermore
c) from about 10-35% by weight, preferably from about 15-30% by weight of water, based on the total composition, and
d) contains at least one linear polydimethylsiloxane which is liquid at about 20° C. in a total quantity of from about 0.005-0.15% by weight, based on the total composition.

All data on the states of aggregation of substances (solid, liquid, gaseous) in this present disclosure refer to normal conditions. “For the purposes of this application, ‘normal conditions’ means, unless otherwise specified, a temperature of about 20° C. and a pressure of about 1013,25 mbar.

As contemplated herein, the C₁₀-C₃₀ alkanecarboxylic acids preferred for the formation of soap gels, which can be substituted with at least one hydroxyl group, are chosen from the saturated C₁₀-C₃₀ alkanecarboxylic acids, in particular decanoic acid, undecanoic acid, dodecanoic acid, 12-hydroxydodecanoic acid, 12-hydroxystearic acid, tridecanoic acid, myristic acid, palmitic acid, stearic acid, isostearic acid, eicosanoic acid and behenic acid, and mixtures thereof. Palmitic acid and stearic acid and mixtures thereof are particularly preferred. Mixtures of palmitic acid and stearic acid in a weight ratio of from about 30:70-70:30 is particularly preferred, preferably from about 40:60-60:40 and especially preferred about 50:50.

The at least one C₁₀-C₃₀ alkane carboxylic acid is present in the compositions as contemplated herein as a sodium salt and is also called soap in this form. Preferred soaps are sodium stearate, sodium isostearate, sodium palmitate, sodium behenate and sodium myristate and mixtures thereof. Sodium stearate, sodium palmitate and mixtures thereof are particularly preferred.

The stick compositions as contemplated herein further contain at least one polyol liquid at about 20° C., chosen from polyvalent C₂-C₉ alkanols with 2-6 hydroxyl groups and polyethylene glycol with 3-10 ethylene oxide units and mixtures thereof, in a total amount of from about 60 to about 80% by weight, based on the total composition.

These polyvalent C₂-C₉ alkanols with 2-6 hydroxyl groups chosen from 1.2-propylene glycol, 2-methyl-1.3-propanediol, glycerol, 1.2-butylene glycol, 1.3-butylene glycol, 1.4-butylene glycol, 1.2-pentanediol, 1.5-pentanediol, 1.6-hexanediol, 1.2.6-hexanetriol, dipropylene glycol, tripropylene glycol, diglycerol and triglycerol, and mixtures of the foregoing substances. Particularly preferred polyethylene glycols with 3-10 ethylene oxide units liquid at about 20° C. are chosen from PEG-3, PEG-4, PEG-6, PEG-7, PEG-8, PEG-9 and PEG-10 and mixtures thereof, whereby PEG-3 to PEG-8 are extremely preferred.

Preferred stick compositions as contemplated herein include at least one polyol liquid at about 20° C., chosen from polyhydric C₂-C₉ alkanols with 2-6 hydroxyl groups and polyethylene glycol with 3-10 ethylene oxide units, chosen from 1.2-propylene glycol, 2-methyl-1,3-propanediol, glycerol, 1.2-butylene glycol, 1.3-butylene glycol, 1.4-butylene glycol, 1.2-pentanediol, 1.5-pentanediol, 1.6-hexanediol, 1.2.6-hexanetriol, dipropylene glycol, tripropylene glycol, diglycerol, triglycerol, PEG-3, PEG-4, PEG-6, PEG-7, PEG-8, PEG-9 and PEG-10 and mixtures of the above substances.

Particularly preferred stick compositions as contemplated herein include at least one polyol liquid at about 20° C., chosen from polyvalent C₂-C₉ alkanols with 2-6 hydroxyl groups and polyethylene glycol with 3-10 ethylene oxide units, in a total amount of from about 65-75% by weight, preferably from about 70-73% by weight, based on the total composition.

The proportion of water in the stick compositions as contemplated herein is from about 10-35% by weight, preferably from about 15-30% by weight, particularly preferably from about 18-25% by weight, extremely preferably from about 20-23% by weight, each based on the total composition.

Stick compositions preferred as contemplated herein contain one or more nonionic oil-in-water emulsifiers in a total amount of from 0 to about 0.3% by weight, based on the total composition, these nonionic oil-in-water emulsifiers being different from glyceryl monoalkyl esters, glyceryl monoalkenyl esters, glyceryl monoalkyl ethers and glyceryl monoalkenyl ethers whose alkyl radical or alkenyl radical has at least 6 carbon atoms.

For the purposes of the present application, surfactants and emulsifiers are amphiphilic (bifunctional) compounds which include at least one hydrophobic and at least one hydrophilic part of the molecule. The hydrophobic radical is preferably a hydrocarbon chain with about 8-28 carbon atoms, which can be saturated or unsaturated, linear, or branched. This C₈-C₂₈ alkyl chain is particularly preferred linear. The basic properties of the surfactants and emulsifiers are oriented absorption at interfaces as well as aggregation to micelles and the formation of lyotropic phases.

Among the non-ionic surfactants suitable as contemplated herein are, for example, polydimethylsiloxanes modified with polyethylene glycol side chains, in particular PEG-12 dimethicones, furthermore addition products of about 6 to 12 ethylene oxide units and one to two propylene oxide units to divalent C10-16 alkane glycols, furthermore alkyl mono- and oligoglycosides corresponding to the formula R⁴O-[G]ₚ, in which R⁴ is an alkyl or alkenyl radical containing about 4 to 22 carbon atoms, G is a sugar unit containing 5 or 6 carbon atoms and p is a number of 1 to 6, further containing about 20-100 mol ethylene oxide per mol ethoxylated castor oil, ethoxylated C₈-C₂₄ alkanols containing about 10-100 mol ethylene oxide per mol, ethoxylated C₈-C₂₄ alkenols containing about 10-100 mol ethylene oxide per mol with about 20-100 moles of ethylene oxide per mole of ethoxylated sorbitan monoesters of linear saturated and unsaturated C₁₂-C₃₀ carboxylic acids, which may be hydroxylated, in particular those of myristic acid, palmitic acid, stearic acid or mixtures of these fatty acids, as well as mixtures of the aforementioned substances.

Nonionic oil-in-water emulsifiers which are chosen from glyceryl monoalkyl esters, glyceryl monoalkenyl esters, glyceryl monoalkyl ethers and glyceryl monoalkenyl ethers whose alkyl radical or alkenyl radical has at least 6 carbon atoms are contained, as contemplated herein, preferably in a total amount of from about 0-2% by weight, particularly preferably from about 0.1-1.5% by weight, extremely preferably from about 0.5-1% by weight, in each case based on the total composition. The above-mentioned compounds act not only as non-ionic oil-in-water emulsifiers, but above all as deodorant agents, which is why their use is preferred, especially for the deodorant stick version, as contemplated herein. A glyceryl monoalkyl ether whose alkyl radical has at least 6 carbon atoms is 2-ethylhexyl glyceryl ether, especially alpha-2-ethylhexyl glyceryl ether. As contemplated herein, extraordinarily preferred stick compositions contain from about 0.1-2 wt. %, preferably from about 0.1-1.5 wt. %, particularly preferably from about 0.5-1 wt. % of 2-ethylhexyl glyceryl ether, alpha-2-ethylhexyl glyceryl ether.

The stick compositions as contemplated herein are further exemplified by a content of at least one linear polymethylsiloxane liquid at about 20° C. in a total amount of from about 0.005-0.15% by weight, preferably from about 0.01-0.10% by weight, particularly preferably from about 0.02-0.08% by weight, extremely preferably from about 0.04-0.07% by weight, in each case based on the total composition.

Preferred embodiments as contemplated herein include the linear polymethylsiloxane liquid at about 20° C. having a kinematic viscosity of from about 10-2000 cSt, measured at about 25° C.

Preferred linear polymethylsiloxanes which are liquid at about 20° C. are chosen from polydimethylsiloxanes with the INCI designation Dimethicone, e.g. available from the product series Dow Corning 200 Fluid with kinematic viscosities of about 10, about 20, about 50, about 100, about 200, about 350, about 500 and about 2000 cSt (about 25° C.) or from the product series Baysilon-Öl M from GE Bayer Silicones.

Further preferred embodiments as contemplated herein include at least one deodorant active substance and/or at least one fragrance.

As contemplated herein, preferred deodorant active ingredients are chosen from antimicrobial, antibacterial, or germ-inhibiting substances, as well as odor adsorbents (e.g. zinc cicinoleate), and mixtures of these active ingredients. Suitable antimicrobial, antibacterial, or germ-inhibiting substances are especially organohalogen compounds as well as organohalides, quaternary ammonium compounds and zinc compounds. Preferred are halogenated phenol derivatives such as hexachlorophene or triclosan (2.4.4′-trichloro-2′-hydroxydiphenyl ether), 3.4.4′-trichlorocarbanilide, chlorhexidine (1.1′-hexamethylene-bis-[5-(4-chlorophenyl)]-biguanide), chlorhexidine gluconate, benzalkonium halides and cetylpyridinium chloride. Other deodorant active ingredients suitable for the present disclosure are chosen from sodium bicarbonate, sodium phenolsulfonate and zinc phenolsulfonate. Other preferred deodorant active ingredients are chosen from 1.2-alkane-(C₆-C₁₈)-diols, glyceryl monoalkyl esters, glyceryl monoalkenyl esters, glyceryl monoalkyl ethers and glyceryl monoalkenyl ethers whose alkyl or alkenyl radical has at least 6 carbon atoms, in particular 2-ethylhexyl glycerol ethers, which are very well tolerated by the skin and mucous membranes and effective against corynebacteria, as well as phenoxyethanol, phenoxyisopropanol (3-phenoxy-propan-2-ol), aniseed alcohol, 2-methyl-5-phenyl-pentan-1-ol, 1,1-dimethyl-3-phenyl-propan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2.2-dimethyl-3-phenylpropan-1-ol, 2.2-dimethyl-3-(3′-methylphenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′.4′-dichlorophenyl)-2-ethylpropan-1-ol, 2-ethyl-3-(2′-methylphenyl)-propan-1-ol, 2-ethyl-3-(4′-methylphenyl)-propan-1-ol, 3-(3′.4′-dimethylphenyl)-2-ethylpropan-1-ol, 2-ethyl-3-(4′-methoxyphenyl)-propan-1-ol, 3-(3′.4′-dimethoxyphenyl)-2-ethylpropan-1-ol, 2-allyl-3-phenylpropan-1-ol and 2-n-pentyl-3-phenylpropan-1-ol, and mixtures of these active substances.

Preferred stick compositions as contemplated herein include at least one deodorant active ingredient is chosen from glyceryl monoalkyl ethers, glyceryl monoalkenyl ethers, glyceryl monoalkyl esters and glyceryl monoalkenyl esters whose alkyl radical or alkenyl radical have at least 6 carbon atoms, in particular 2-ethylhexylglycerol ether, furthermore hexachlorophenes, triclosan (2,4,4′-trichlor-2′-hydroxydiphenylether), 3.4.4′-trichlorocarbanilide, chlorhexidine (1.1′-hexamethylene-bis-[5-(4-chlorophenyl)]-biguanide), chlorhexidine gluconate, benzalkonium halides, cetylpyridinium chloride, sodium bicarbonate, Sodium phenol sulfonate, zinc phenol sulfonate, 1.2-alkane (C₆-C₁₈) diols, phenoxyethanol, phenoxyisopropanol (3-phenoxypropan-2-ol), anise alcohol, 2-methyl-5-phenylpentan-1-ol, 1,1-Dimethyl-3-phenyl-propan-1-ol, benzyl alcohol, 2-phenylethan-1-ol, 3-phenylpropan-1-ol, 4-phenylbutan-1-ol, 5-phenylpentan-1-ol, 2-Benzylheptan-1-ol, 2.2-dimethyl-3-phenylpropan-1-ol, 2.2-dimethyl-3-(3′-methylphenyl) propan-1-ol, 2-ethyl-3-phenylpropan-1-ol, 2-ethyl-3-(3′-methylphenyl) propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′, 4′-dichlorophenyl)-2-ethylpropan-1-ol, 2-ethyl-3-(2′-methylphenyl) propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3rd′, 4′-Dimethoxyphenyl)-2-ethylpropan-1-ol, 2-allyl-3-phenylpropan-1-ol and 2-n-pentyl-3-phenylpropan-1-ol, as well as mixtures of these active ingredients.

Preferred stick compositions as contemplated herein include at least one deodorant active substance in a total amount of from about 0.1-10% by weight, preferably from about 0.2-7% by weight, particularly preferably from about 0.3-5% by weight and extremely preferably from about 0.4-1.0% by weight, based on the total weight of the active substance in the total composition.

The definition of a fragrance within the meaning of the present disclosure corresponds to the usual professional definition as it can be found in the RÖMPP Chemie Lexikon, December 2007. According to this, a fragrance is a chemical compound with smell and/or taste that excites the receptors of the hair cells of the olfactory system (adequate stimulus). The physical and chemical properties required for this are a low molar mass of maximum 300 g/mol, high vapor pressure, minimal water, and high lipid solubility as well as weak polarity and the presence of at least one osmophoric group in the molecule. In order to distinguish volatile, low-molecular substances which are normally, and also for the purposes of the present application, not considered and used as perfumes but primarily as solvents, such as ethanol, propanol, isopropanol and acetone, from perfumes of the present disclosure, perfumes of the present disclosure have a molecular weight of from about 74 to about 300 g/mol, contain at least one osmophoric group in the molecule and have an odor and/or taste, that is to say, they excite the receptors of the hair cells of the olfactory system. Examples of fragrance and aromatic compounds of the ester type are benzyl acetate, phenoxyethyl isobutyrate, p-tert. butylcyclohexyl acetate, linalyl acetate, dimethylbenzylcarbinyl acetate (DMBCA), phenylethyl acetate, benzyl acetate, ethylmethylphenylglycinate, allylcyclohexyl propionate, styrenallylpropionate, benzyl salicylate, cyclohexyl salicylate, floramate, melusate and jasmecyclate. Examples of fragrance and aromatic compounds of the ether type are benzyl ethyl ether and ambroxane, examples of fragrance and aromatic compounds of the aldehyde type are the linear alkanals with 8-18 C atoms, citral, citronellal, citronellyloxy-acetaldehyde, cyclamenaldehyde, lily and bourgeonal; examples of odoriferous compounds of the ketone type are jonones, alpha-isomethylionone and methylcedryl ketone; examples of odoriferous compounds of the alcohol type are anethole, citronellol, eugenol, geraniol, linalool, phenylethyl alcohol and terpineol; examples of odoriferous compounds of the terpene type are limonene and pinene. Examples of fragrance and scent compounds are pine, citrus, jasmine, patchouli, rose, ylang ylang oil, muscatel sage oil, chamomile oil, clove oil, mint oil, cinnamon leaf oil, lime blossom oil, juniper berry oil, vetiver oil, olibanum oil, galbanum oil, labdanum oil, orange blossom oil, neroli oil, orange peel oil and sandalwood oil, furthermore the essential oils like angelica root oil, anise oil, arnica blossom oil, basil oil, bay oil, bergamot oil, champaca blossom oil, silver fir oil, silver fir cone oil, elemi oil, eucalyptus oil, fennel oil, spruce needle oil, geranium oil, ginger grass oil, guaiac wood oil, gurjun balsam oil, helichrysum oil, ho-oil, ginger oil, Iris oil, cajeput oil, calamus oil, camomile oil, camphor oil, canaga oil, cardamom oil, cassia oil, pine needle oil, copaïva balsam oil, coriander oil, spearmint oil, caraway oil, cumin oil, lavender oil, lemongrass oil, lime oil, tangerine oil, lemon balm oil, musk seed oil, myrrh oil, clove oil, niaouli oil, orange oil, origanum oil, Palmarosa oil, patchouli oil, Peru balsam oil, petitgrain oil, pepper oil, peppermint oil, pimento oil, pine oil, rose oil, rosemary oil, sandalwood oil, celery oil, spik oil, star anise oil, turpentine oil, thuja oil, thyme oil, verbena oil, juniper berry oil, wormwood oil, wintergreen oil, hyssop oil, cinnamon oil, citronella oil, lemon oil and cypress oil. Other fragrance and aroma compounds are ambrettolide, alpha-amyl cinnamaldehyde, anethole, anisaldehyde, anise alcohol, anisole, methyl anthranilic acid ester, acetophenone, benzyl acetone, Benzaldehyde, ethyl benzoate, benzophenone, benzyl alcohol, benzyl acetate, benzyl benzoate, benzyl formate, benzyl valerianate, borneol, bornyl acetate, alpha-bromostyrene, n-decylaldehyde, n-dodecyl aldehyde, eugenol, eugenol methyl ether, eucalyptol, farnesol, fenchone, fenchyl acetate, geranyl acetate, geranyl formate, heliotropin, methyl heptanecarboxylate, heptaldehyde, Hydroquinone dimethyl ether, hydroxycinnamic aldehyde, hydroxycinnamic alcohol, indole, iron, isoeugenol, isoeugenol methyl ether, isosafrol, jasmone, camphor, carvacrol, carvone, p-cresol methyl ether, coumarin, p-methoxyacetophenone, methyl n-amyl ketone, methyl anthranilic acid methyl ester, p-methylacetophenone, methyl chavicol, p-methyl quinoline, methyl beta-naphthyl ketone, methyl n-nonyl acetaldehyde, Methyl-n-nonylketone, muscone, beta-naphthol ethyl ether, beta-naphthol methyl ether, nerol, nitrobenzene, n-nonylaldehyde, nonyl alcohol, n-octylaldehyde, p-oxy-acetophenone, pentadecanolide, beta-phenylethyl alcohol, phenylacetaldehyde dimethyacetal, phenylacetic acid, pulegon, safrole, isoamyl salicylic acid ester, methyl salicylic acid ester, hexyl salicylic acid ester, cyclohexyl salicylic acid ester, Santalol, skatole, terpineol, thymene, thymol, gamma-undecalactone, vanillin, veratrum aldehyde, cinnamic aldehyde, cinnamic alcohol, cinnamic acid, ethyl cinnamate and benzyl cinnamate.

Other (more volatile) fragrances are alkyl isothiocyanates (alkyl legumes), butanedione, limonene, linalool, linayl acetate and propionate, menthol, menthone, methyl-n-heptenone, phellandrene, phenylacetaldehyde, terpinyl acetate, citral and citronellal.

Preferably mixtures of different fragrances are used, which together create an appealing scent.

Suitable perfume oils may also contain natural fragrance mixtures as available from vegetable or animal sources, e.g. pine, citrus, jasmine, rose, lily, or ylang-ylang oil. Essential oils of lower volatility, which are mostly used as aroma components, are also suitable as perfume oils, e.g. sage oil, camomile oil, melissa oil, mint oil, cinnamon leaf oil, lime blossom oil, juniper berry oil, vetiver oil, olive oil, galbanum oil, laudanum oil, clove oil, iso-eugenol, thyme oil, bergamot oil, geranium oil and rose oil.

Preferred stick compositions as contemplated herein include at least one fragrance in a total amount of from about 0.1-15% by weight, preferably from about 0.5-10% by weight, particularly preferably from about 1-8% by weight, extremely preferably from about 2-7% by weight, further extremely preferably from about 3-6% by weight, each based on the total weight of the composition.

Particularly preferred designs as contemplated herein are exemplified by the fact that no ethanol is contained. Ethanol-free compositions are particularly skin-friendly but may have the disadvantage of foaming more during production, which can affect the filling process and/or product quality.

Further stick compositions preferred as contemplated herein include zero to about 3 wt. %, particularly preferably from about 0-1 wt. % ethanol, based on the total composition.

Further stick compositions preferred as contemplated herein include additionally at least one polypropylene glycol with from about 20-35 propylene oxide units, preferably with from about 24-30 propylene oxide units, extremely preferably with about 26 propylene oxide units (PPG-26), preferably in a total amount of from about 0.01-0.3% by weight, particularly preferably from about 0.04-0.2% by weight, extremely preferably from about 0.1-0.15% by weight, in each case based on the total weight of the composition. Surprisingly, it was found that small amounts of polypropylene glycol with from about 20-35 propylene oxide units further enhance the foam-inhibiting effect of the linear liquid polydimethylsiloxane and thus allow the filling process to be further accelerated.

Further stick compositions preferred as contemplated herein include at least one polyethylene glycol/polypropylene glycol copolymer, which preferably has 15-25 ethylene oxide units and from about 40-80 propylene oxide units in the molecule. PEG/PPG-20/60 copolymer is particularly preferred. The at least one polyethylene glycol/polypropylene glycol copolymer, which preferably has 15-25 ethylene oxide units and from about 40-80 propylene oxide units in the molecule and is particularly preferably PEG/PPG-20/60 copolymer, is preferably contained in a total amount of from about 0.01-0.3% by weight, particularly preferably from about 0.02-0.15% by weight, extremely preferably from about 0.07-0.1% by weight, each based on the total weight of the composition. Surprisingly, it was found that small amounts of polyethylene glycol/polypropylene glycol copolymer or polyethylene glycol/polypropylene glycol copolymers further enhance the foam-inhibiting effect of the linear liquid polydimethylsiloxane and thus allow the filling process to be further accelerated.

Further stick compositions preferred as contemplated herein include additional trimethylsiloxysilicate/dimethicone crosspolymer, preferably in a total amount of from about 0.001-0.02% by weight, particularly preferably from about 0.005-0.01% by weight, each based on the total weight of the composition. Surprisingly, it was found that small amounts of trimethylsiloxysilicate/dimethicone crosspolymer further enhance the anti-foaming effect of the linear liquid polydimethylsiloxane and thus allow to further accelerate the filling process.

Also disclosed is a non-therapeutic cosmetic process for the care and/or deodorisation of the skin, in particular the skin in the axillary region, a composition according to one of the Claims is applied to the skin, in particular to the skin in the axillary region.

Further disclosed is a process for preparing a composition according to one of the claims, wherein

a) the sodium salt of at least one C₁₀-C₃₀ alkanecarboxylic acid, which may be substituted with at least one hydroxyl group, in a total amount of from about 5-7% by weight, based on the total composition, further
b) at least one polyol liquid at about 20° C., chosen from polyvalent C₂-C₉ alkanols with 2-6 hydroxyl groups and polyethylene glycol with 3-10 ethylene oxide units and mixtures thereof, in a total amount of from about 60 to about 80% by weight, based on the total composition, furthermore
c) from about 10-35% by weight, preferably from about 15-30% by weight of water, based on the total composition, and
d) contains at least one linear polydimethylsiloxane which is liquid at about 20° C. in a total quantity of from about 0.005-0.15% by weight, based on the total are mixed while heated to from about 70-90° C. and then filled into suitable pencil molds where they are statically cooled.
As contemplated herein, this process can be used to produce single-phase as well as multi-phase or multi-layer pen compositions based on the core-shell principle.

The following examples are intended to illustrate the subject matter of the present disclosure, without restricting it thereto.

TABLE 1
Example recipes (all quantities in weight %)

| Raw material | V1 | E1 | E2 | E3 | E4 |
|---|---|---|---|---|---|
| Sodium stearate | 6.00 | 6.00 | 6.00 | 6.00 | 6.00 |
| Dipropylene glycol | 35.93 | 35.93 | 35.93 | 35.93 | 35.93 |
| 1.2-propanediol | 35.93 | 35.93 | 35.93 | 35.93 | 35.93 |
| Phenoxyethanol, pure | 0.90 | 0.90 | 0.90 | 0.90 | 0.90 |
| Perfume | 1.00 | 1.00 | 1.00 | 1.00 | 1.00 |
| Dimethicone | — | 0.02 | 0.04 | 0.08 | 0.01 |
| (polydimethylsiloxane | | | | | |
| liquid at 20° C.) | | | | | |
| PPG-26 | — | 0.05 | 0.10 | 0.20 | 0.025 |
| PEG/PPG-20/60 | — | 0.02 | 0.04 | 0.08 | 0.01 |
| Copolymer | | | | | |
| Trimethylsiloxysilicate/ | | 0.002 | 0.004 | 0.008 | 0.001 |
| Dimethicone | | | | | |
| Crosspolymer | | | | | |
| Water, fully desalinated | 20.24 | 20.148 | 20.056 | 19.872 | 20.194 |
| Foaming behavior from | 1 | 4 | 5 | 5 | 3 |
| 1-5 (1: much foam, | | | | | |
| 5: no foam) | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic stick composition in the form of a gel based on a fatty acid salt and comprising
a) the sodium salt of at least one C₁₀-C₃₀ alkanecarboxylic acid, which may be substituted with at least one hydroxyl group, in a total amount of from about 5-7% by weight, based on the total weight of the composition,
b) at least one polyol that is liquid at about 20° C., chosen from polyvalent C₂-C₉ alkanols with about 2-6 hydroxyl groups and polyethylene glycol with about 3-10 ethylene oxide units and mixtures thereof, in a total amount of from about 60 to about 80% by weight, based on the total weight of the composition,
c) from about 10-35% by weight of water, based on the total weight of the composition, and
d) at least one linear polydimethylsiloxane which is liquid at about 20° C. in a total quantity of from about 0.005-0.15% by weight based on the total weight of the composition.

2. Stick composition according to claim 1, wherein the at least one linear polydimethylsiloxane which is liquid at about 20° C. is included in a total amount of from about 0.01-0.10% by weight based on the total weight of the composition.

3. Stick composition according to claim 1, further comprising at least one deodorant active substance and/or at least one fragrance substance.

4. Stick composition according to claim 1, wherein one or more nonionic oil-in-water emulsifiers are included in a total amount of from 0 to about 0.3% by weight, based on the total weight of the composition, wherein the nonionic oil-in-water emulsifiers are different from glyceryl monoalkyl esters, glyceryl monoalkenyl esters, glyceryl monoalkyl ethers and glyceryl monoalkenyl ethers whose alkyl radical or alkenyl radical has at least 6 carbon atoms.

5. Stick composition according to claim 1, wherein a mixture of 1,2-propanediol and dipropylene glycol is included as the polyol b) that is liquid at about 20° C.

6. Stick composition according to claim 5, wherein the mixture of 1.2-propanediol and dipropylene glycol comprises of from about 20-80% by weight of dipropylene glycol based on a total weight of the mixture.

7. Stick composition according to claim 1, wherein from 0 to about 3% by weight of ethanol, based on the total weight of the composition, is included.

8. Stick composition according to claim 1, wherein the C₁₀-C₃₀ alkanecarboxylic acid is chosen from decanoic acid, undecanoic acid, dodecanoic acid, 12-hydroxydodecanoic acid, 12-hydroxystearic acid, tridecanoic acid, myristic acid, palmitic acid, stearic acid, isostearic acid, Eicosanoic acid, behenic acid, and mixtures thereof.

9. Stick composition according to claim 2, wherein the at least one deodorant active ingredient is chosen from glyceryl monoalkyl ethers, glyceryl monoalkenyl ethers, glyceryl monoalkyl esters and glyceryl monoalkenyl esters whose alkyl radical or alkenyl radical has at least 6 carbon atoms and mixtures thereof.

10. Stick composition according to claim 1, wherein at least one nonionic oil-in-water emulsifier are included, are chosen from glyceryl monoalkyl esters, glyceryl monoalkenyl esters, glyceryl monoalkyl ethers and glyceryl monoalkenyl ethers whose alkyl radical or alkenyl radical has at least 6 carbon atoms, and are present in a total amount of from about 0.1-2% by weight of the total weight of the composition.

11. Stick composition according to claim 1, wherein additionally at least one polypropylene glycol with from about 20-35 propylene oxide units, is included in the composition in an amount of from about 0.01-0.3% by weight based on the total weight of the composition.

12. Stick composition according to claim 1, wherein additionally at least one polyethylene glycol/polypropylene glycol copolymer is included.

13. Stick composition according to claim 1, wherein additionally at least one trimethylsiloxysilicate/dimethicone crosspolymer is included.

14. Stick composition according to claim 1, wherein the at least one linear polydimethylsiloxane which is liquid at about 20° C. is included in a total amount of from about 0.04-0.07% by weight based on the total weight of the composition.

15. Stick composition according to claim 14, further comprising at least one deodorant active substance and/or at least one fragrance substance.

16. Stick composition according to claim 15, wherein one or more nonionic oil-in-water emulsifiers are included in a total amount of from 0 to about 0.3% by weight, based on the total weight of the composition, wherein the nonionic oil-in-water emulsifiers are different from glyceryl monoalkyl esters, glyceryl monoalkenyl esters, glyceryl monoalkyl ethers and glyceryl monoalkenyl ethers whose alkyl radical or alkenyl radical has at least 6 carbon atoms.

17. Stick composition according to claim 16, wherein a mixture of 1,2-propanediol and dipropylene glycol is included as the polyol b) that is liquid at about 20° C.

18. Stick composition according to claim 17, wherein the mixture of 1.2-propanediol and dipropylene glycol comprises of from about 60-40% by weight of dipropylene glycol based on a total weight of the mixture.

19. A cosmetic stick composition in the form of a gel based on a fatty acid salt and comprising:
a sodium salt of a mixture of palmitic acid and stearic acid in a weight ratio of about 50:50 and present in a total amount of 5-7% by weight, based on the total composition;
a mixture of 1,2-propanediol and dipropylene glycol comprising 60-40% by weight of dipropylene glycol and present in a total amount of 60 to 80% by weight based on the total weight of the composition;
15-30% by weight of water based on the total weight of the composition;
at least one linear polydimethylsiloxane which is liquid at 20° C. in a total quantity of about 0.04-0.07% by weight based on the total weight of the composition;
one or more nonionic oil-in-water emulsifiers that are chosen from glyceryl monoalkyl esters, glyceryl monoalkenyl esters, glyceryl monoalkyl ethers and glyceryl monoalkenyl ethers whose alkyl radical or alkenyl radical has at least 6 carbon atoms and present in a total amount of about 0.5-1% by weight based on the total weight of the composition;
at least one deodorant active substance and/or at least one fragrance substance chosen from 2-ethylhexyl glycerol ethers, hexachlorophene, triclosan (2,4,4′-trichloro-2′-hydroxydiphenyl ether), 3,4,4′-trichlorocarbanilide, chlorhexidine (1,1′-hexamethylene-bis-15-(4-chlorophenyl)]-biguanide), chlorhexidine gluconate, benzalkonium halides, cetylpyridinium chloride, sodium bicarbonate, sodium phenolsulphonate, zinc phenolsulphonate, 1,2-alkane-(C₆-C₁₈)-diols, phenoxyethanol, phenoxyisopropanol (3-phenoxy-propan-2-ol), aniseed alcohol, 2-methyl-5-phenyl-pentan-1-ol, 1, 1-dimethyl-3-phenyl-propan-1-ol, benzyl alcohol, 2-phenylethan-1-ol, 3-phenylpropan-1-ol, 4-phenylbutan-1-ol, 5-phenylpentan-1-ol, 2-benzylheptan-1-ol, 2.2-dimethyl-3-phenylpropan-1-ol, 2.2-dimethyl-3-(3′-methylphenyl)-propan-1-ol, 2-ethyl-3-phenylpropan-1-ol, 2-ethyl-3-(3′-methylphenyl)-propan-1-ol, 3-(3′-chlorophenyl)-2-ethylpropan-1-ol, 3-(2′-chlorophenyl)-2-ethylpropan-1-ol, 3-(4′-chlorophenyl)-2-ethylpropan-1-ol, 3-(3′,4′-dichlorophenyl)-2-ethylpropan-1-oll, 2-ethyl-3-(2′-methylphenyl)-propan-1-ol, 2-ethyl-3-(4′-methylphenyl)-propan-1-ol, 3-(3′,4′-dimethylphenyl)-2-ethylpropan-1-ol, 2-ethyl-3-(4′-methoxyphenyl)-propan-1-ol, 3-(3′,4′-dimethoxyphenyl)-2-ethylpropan-1-ol, 2-allyl-3-phenylpropan-1-ol and 2-n-pentyl-3-phenylpropan-1-ol, and mixtures of these active substances;
at least one polypropylene glycol with 26 propylene oxide units in a total amount of about 0.1-0.15% by weight based on the total weight of the composition;
at least one polyethylene glycol/polypropylene glycol copolymer having about 15-25 ethylene oxide units and about 40-80 propylene oxide units in the molecule in an amount of about 0.07-0.1% by weight based on the total weight of the composition; and
at least one trimethylsiloxysilicate/dimethicone crosspolymer in an amount of about 0.005-0.01% by weight based on the total weight of the composition.
